(19) United States
(12) Patent Application Publication
(10) Pub. No.: US 2009/0291872 A1
(43) Pub. Date: Nov. 26, 2009

(54) Ionic Liquids and Methods For Using the Same

(75) Inventors: Jason E. Bara, Denver, CO (US); Dean E. Camper, Superior, CO (US); Douglas L. Gin, Longmont, CO (US); Richard D. Noble, Boulder, CO (US)

Correspondence Address: COOLEY GODWARD KRONISH LLP;ATTN: Patent Group, Suite 1100, 777 - 6th Street, NW, WASHINGTON, DC, 20001, US

(73) Assignee: The Regents of the University of Colorado, Denver, CO (US)

(21) Appl. No.: 12/333,115

(22) Filed: Dec. 11, 2008

Related U.S. Application Data
(60) Provisional application No. 61/055,135, filed on May 21, 2008.

(51) Int. Cl. B01D 53/44; B01D 53/46; B01D 53/56; B01D 53/62; C07C 7/10; C11D 3/30

(52) U.S. Cl. 510/175; 423/210; 423/228; 252/184; 423/246; 423/243.01; 423/235; 423/219; 423/245.2; 585/860; 510/499

(57) ABSTRACT

The present invention provides compositions comprising ionic liquids and an amine compound, and methods for using and producing the same. In some embodiments, the compositions of the invention are useful in reducing the amount of impurities in a fluid medium or a solid substrate.

### CROSS-REFERENCE TO RELATED APPLICATIONS

This application claims the priority benefit of U.S. Provisional Application No. 61/055,135, filed May 21, 2008, which is incorporated herein by reference in its entirety.

### STATEMENT REGARDING FEDERALLY FUNDED RESEARCH

The U.S. Government has a paid-up license in this invention and the right in limited circumstances to require the patent owner to license others on reasonable terms as provided for by the terms of Grant Nos. AB07CBT010 and HDTRA1-08-1-0028 awarded by U.S. Army Research Office and Grant No. DMR-0552399 awarded by the National Science Foundation.

### FIELD OF THE INVENTION

The present invention relates to compositions comprising ionic liquids and an amine compound, and methods for using and producing the same. In some embodiments, the compositions of the invention are useful in reducing the amount of impurities in a fluid medium or a solid substrate.

### BACKGROUND OF THE INVENTION

Ionic liquids are “green” materials with great potential to replace the volatile organic solvents used throughout industrial and laboratory settings. An ionic liquid is a liquid that contains essentially only ions. Some ionic liquids, such as ethylammonium nitrate are in a dynamic equilibrium where at any time more than 99.99% of the liquid is made up of ionic rather than molecular species. The term “ionic liquid” is commonly used for salts whose melting point is relatively low (e.g., below 100° C.). The salts that are liquid at room temperature are called room-temperature ionic liquids, or RTILs. RTILs possess obvious advantages over traditional solvents when considering user safety and environmental impact. Under many conditions, RTILs have negligible vapor pressures, are largely inflammable, and exhibit thermal and chemical stability. However, it is the ability to tailor the chemistry and properties of an RTIL solvent in a variety of ways that provide more useful features, for example, modifying the ionic liquid to modulate the solubility of an amine compound and/or the impurity.

Improved and highly efficient separations of “light” gases (e.g., CO₂, O₂, N₂, CH₄, H₂, and hydrocarbons) are important as fuel use, demand, and costs rise. RTILs have been investigated in other energy-intensive technologies, such as amine scrubbing, for the capture of “acid” gases (CO₂, H₂S, SO₂, etc.). The presence of acid gases in many natural gas fields around the world negatively impacts the quality and viability of those sources.

Recently there has been great interest in CO₂ capture and sequestration, stemming from the immediate need to reduce greenhouse gas emissions. It is estimated that cuts of over 60% would be needed stabilize the climate. Most CO₂ capture studies are currently looking at capturing CO₂ at atmospheric pressures from coal or gas-fired gas plants. The most viable method, in the near-term, to accomplish this post-combustion capture is through chemical absorption, a process where there is substantial room for improvement.

While CO₂ removal from natural gas is useful to the increase in the energy content per volume of natural gas and reduce pipeline corrosion. H₂S removal is important because it is extremely harmful and can even be lethal. H₂S combustion leads to the formation of SO₂, another toxic gas and a component leading to acid rain. Amine-based “scrubbing” is used in 95% of U.S. natural gas “sweetening” operations. In this process, CO₂ (and H₂S) react with amines to form an aqueous carbamate. CO₂ (and H₂S) can be released if the solution is heated and/or the partial pressure reduced.

Generally, the capture of acid gases from natural gas is performed at higher pressures than from post-combustion processes. Typically, the capture pressure is greater than 1 atm, and often at least about 6 atm. In some cases, the type of amine effective in a given application is related to the partial pressure of the acid gas in the stream with primary (1°) alkanolamines (e.g., MEA), secondary (2°) (e.g., diethanolamine (DEA)), and tertiary (3°) (e.g., triethanolamine (TEA)) being suited for low, moderate and high pressures, respectively. In some instances, tertiary amines can also separate H₂S from CO₂. While the amine-based scrubbing process is effective for separating CO₂ from other gases, it is energy-intensive.

Accordingly, there is a need for a method that is more energy efficient in removing impurities or undesired substances from a fluid medium.

### SUMMARY OF THE INVENTION

Some aspects of the invention relate to compositions and methods for reducing or removing an impurity and/or undesired material from a source. In some embodiments, an impurity removing mixture is provided to reduce the amount of impurity from a fluid medium or a solid substrate. The impurity removing mixture typically comprises an ionic liquid and an amine compound. The impurity removing mixture can also include a solvent, typically an organic solvent. In some embodiments, the ionic liquid is a room temperature ionic liquid. The method generally involves contacting the impurity removing mixture with a source under conditions sufficient to remove the impurity from the source. Without being bound by any theory, it is believed that typically the impurity form a complex or an addition product with the amine compound. In some instances, it is believed that the ionic liquid solubilizes the impurity.

In some embodiments, the complex or the addition product forms a precipitate.

Still in other embodiments, the amine compound comprises a monoamine compound of the formula:

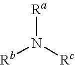

or a diamine compound of the formula:

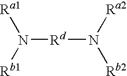

where
each of R^{a}, R^{a1}, R^{a2}, R^{b}, R^{b1}, and R^{b2} is independently hydrogen, alkyl, aryl, aralkyl, cycloalkyl, haloalkyl, heteroalkyl, alkenyl, alkynyl, silyl or siloxyl;
R^{c} is hydrogen, alkyl, aryl, aralkyl, cycloalkyl, haloalkyl, heteroalkyl, alkenyl, alkynyl, silyl, siloxyl, or a nitrogen protecting group; and p1 R^{d} is alkylene, arylene, aralkylene, cycloalkylene, haloalkylene, heteroalkylene, alkenylene, alkynylene, silylene or siloxylene.

In some embodiments the heteroalkyl is a hydroxyalkyl group. In some particular instances, the monoamine compound is selected from the group consisting of mono(hydroxyalkyl)amine, di(hydroxyalkyl)amine, tri(hydroxyalkyl)amine, and a combination thereof. In some particular cases, the monoamine compound comprises monoethanolamine, diglycolamine, diethanolamine, diisopropanolamine, triethanolamine, methyldiethanolamine or a combination thereof.

Still in other embodiments, the impurity removing mixture comprises an organic solvent, water, or a combination thereof. Typically, an organic solvent is used.

Yet in other embodiments, the IL is an imidazolium-based RTIL. Within theses embodiments, in some instances the imidazolium-based RTIL is of the formula:

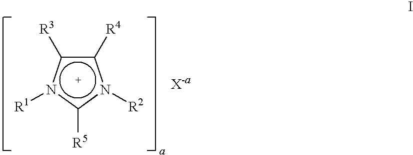

where
a is an oxidation state of X;
X is a counter anion; and
each of R¹ and R² is independently alkyl, heteroalkyl, cycloalkyl, haloalkyl, silyl, siloxyl, aryl, alkenyl, or alkynyl;
each of R³, R⁴, and R⁵ is independently hydrogen, alkyl, cycloalkyl, heteroalkyl, haloalkyl, silyl, siloxyl, aryl, alkenyl, or alkynyl.

Within the imidazolium-based RTIL of Formula I, in some instances X comprises OTf, BF₄, PF₆, Tf₂N, halide, dicyanamide (dca), alkyl sulfonate (e.g., mesylate) or aromatic sulfonate (e.g tosylate).

In other embodiments, the impurity comprises CO₂, CO, COS, H₂S, SO₂, NO, N₂O, mercaptans (e.g., alkylmercaptans), H₂O, O₂, H₂, N₂, methane, propane, and other relatively short chain hydrocarbons, other volatile organic compounds, or a combination thereof.

Other aspects of the invention provide a method for reducing the amount of one or more impurities from a gaseous emission stream, said method comprising contacting the gaseous emission stream with an impurity removing mixture comprising:
an ionic liquid; and
an amine compound,
   under conditions sufficient to reduce the amount of one or more impurities from the gaseous emission stream.

In some embodiments, the impurity removing mixture further comprises a solvent. Within these embodiments, the solvent can be one or more of different ionic liquids, an organic solvent, water, or a mixture thereof. Typically, the solvent is an organic solvent.

Still in other aspects of the invention provide a composition comprising an ionic liquid (IL) and a heteroalkylamine compound. Typically, the ionic liquid is a room temperature ionic liquid (RTIL). In some embodiments, the ionic liquid comprises an imidazole core structure moiety. Still in other embodiments, the heteroalkylamine compound is an alkanolamine compound.

Yet other aspects of the invention provide a composition comprising an ionic liquid and an amine compound, wherein the relative volume % of said ionic liquid compared to the total volume of said ionic liquid and said amine compound is about 60 vol % or less. In some embodiments, the relative volume % of said ionic liquid is about 50 vol % or less. Still in other embodiments, the amine compound is a heteroalkylamine compound. Often the heteroalkylamine compound is an alkanolamine compound.

Other aspects of the invention provide a method for removing or reducing the amount of an impurity from a fluid medium to produce a purified fluid stream. The method generally comprises contacting the fluid medium with an impurity removing mixture under conditions sufficient to remove the impurity from the fluid medium to produce a purified fluid stream. Typically, the impurity removing mixture comprises an ionic liquid, and an amine compound. In some embodiments, the impurity removing mixture further comprises a solvent. Often the solvent is an organic solvent. Still in other embodiments, the ionic liquid is a room temperature ionic liquid (RTIL). In some embodiments, the impurity to be removed is selected from the group consisting of CO₂, CO, COS, H₂S, SO₂, NO, N₂O, H₂O, O₂, H₂, N₂, C₁-C₈ hydrocarbon, a volatile organic compound, and a combination thereof. In some instances, the volatile organic compound comprises an organothiol compounds, hydrocarbon, or a mixture thereof. Often the impurity to be removed or reduced comprises CO₂, SO₂, H₂S, or a combination thereof. In other embodiments, the amine compound comprises a heteroalkylamine compound. Often the heteroalkylamine compound comprises alkanolamine compound. Still in other embodiments, the relative volume % of the ionic liquid relative to the total volume of ionic liquid and the amine compound is about 60 vol % or less. Yet in other embodiments, the step of contacting the fluid medium with the impurity removing mixture is conducted under pressure, e.g., greater than 1 atm. When the fluid medium is contacted with the impurity removing mixture under pressure, typically a pressure of at least about 6 atm is used, often at least about 8 atm, and more often at least about 10 atm. In some embodiments, the fluid medium comprises a hydrocarbon source. Often the hydrocarbon source comprises natural gas, oil, or a combination thereof. Still in other embodiments, the step of contacting the fluid medium with the impurity removing mixture produces an addition product or a complex between the impurity and the amine compound.

Yet other aspects of the invention provide a method for removing an impurity from a solid substrate surface to produce a clean solid substrate surface. The method typically comprises contacting the solid substrate surface with an impurity removing mixture under conditions sufficient to remove the impurity from the solid substrate surface to produce a clean solid substrate surface. The impurity removing mixture typically comprises an ionic liquid and an amine compound. In some embodiments, the solid substrate comprises a semi-conductor.

### BRIEF DESCRIPTION OF THE DRAWINGS

FIG. 1 is a schematic representation of a typical aqueous amine gas treatment unit;

FIG. 2 is a graph of CO₂ pressure data for uptake in an equimolar compound 2a-MEA solution;

FIG. 3 is a graph of CO₂ conversion to MEA-carbamate as a function of time;

FIG. 4 is a plot of the release of CO₂ from MEA-carbamate in compound 2a at 100° C. under reduced pressure as a function of time;

FIG. 5 is a graph showing increased CO₂ uptake in compound 2b-DEA at 100° C. with increasing pressure of CO₂;

FIG. 6 is a plot of Average natural log of the Henry's constant versus average measured mixture molar volume to the −4/3 power at 40° C., where the lines represent the RST models (eq 6) for each gas;

FIG. 7A is a plot of solubility selectivity versus average measured molar volume of the IL at 40° C. for CO₂ with N₂, where the lines represent the RST model prediction;

FIG. 7B is a plot of solubility selectivity versus average measured molar volume of the IL at 40° C. for CO₂ with CH₄, where the lines represent the RST model prediction.

FIG. 8A is a plot of gas loading at 1 atm and 40° C. as a function of molar volume for CO₂, where the line represents the RST model developed from pure RTIL solubility data.

FIG. 8B is a plot of gas loading at 1 atm and 40° C. as a function of molar volume for N₂, where the line represents the RST model developed from pure RTIL solubility data.

FIG. 8C is a plot of gas loading at 1 atm and 40° C. as a function of molar volume for CH₄, where the line represents the RST model developed from pure RTIL solubility data.

FIG. 9 is a graph showing the relationship between the carbamate precipitation point vs. vol % of IL compound.

### DETAILED DESCRIPTION OF THE INVENTION

### Definitions

Unless the context requires otherwise, the terms “sequestration,” “reduction,” “removal,” and “separation” are used interchangeably herein and refer generally to techniques or practices whose partial or whole effect is to reduce the amount of or remove one or more impurities or undesired substances from a given material (e.g., a fluid medium or a solid substrate) such as gas mixtures, gas sources or point emissions sources. In some embodiments, the removed impurity and/or undesired substance (hereinafter collectively “impurity” or “impurities” unless the context requires otherwise) are stored in some form or another so as to prevent its release. Use of these terms do not exclude any form of the described embodiments from being considered impurity and/or undesired substance “sequestration,” “reduction,” “separation,” or “removal” techniques.

Unless the context requires otherwise, the terms “impurity” and “undesired material” are used interchangeably herein and refer to a substance within a liquid, gas, or solid, which differs from the desired chemical composition of the material or compound. Impurities are either naturally occurring or added during synthesis of a chemical or commercial product. During production, impurities may be purposely, accidentally, inevitably, or incidentally added into the substance or produced or it may be present from the beginning.

The term “undesired substance” refers to a substance that is present within a liquid, gas, or solid that one wishes to reduce the amount of or eliminate completely.

The term “acid gas” refers to any gas that reacts with a base. Some acid gases form an acid when combined with water and some acid gases have an acidic proton (e.g., pKₐ of less than that of water or pKₐ of about 14). Exemplary acid gases include, but are not limited to, carbon dioxide, hydrogen sulfide (H₂S), COS, sulfur dioxide (SO₂), and the like.

“Alkyl” refers to a saturated linear monovalent hydrocarbon moiety of one to twenty, typically one to twelve and often one to six, carbon atoms or a saturated branched monovalent hydrocarbon moiety of three to twenty, typically three to twelve and often three to six, carbon atoms. Exemplary alkyl group include, but are not limited to, methyl, ethyl, n-propyl, 2-propyl, tert-butyl, pentyl, hexyl and the like.

“Alkylene” refers to a saturated linear saturated divalent alkyl moiety defined above. Exemplary alkylene groups include, but are not limited to, methylene, ethylene, propylene, butylene, pentylene, hexylene, and the like.

“Alkenyl” refers to a linear monovalent hydrocarbon moiety of two to twenty, typically two to twelve and often two to six, carbon atoms or a branched monovalent hydrocarbon moiety of three to twenty, typically three to twelve and often three to six carbon atoms, containing at least one carbon-carbon double bond. Exemplary alkenyls include, but are not limited to, ethenyl, propenyl, and the like.

“Alkynyl” refers to a linear monovalent hydrocarbon moiety of two to twenty, typically two to twelve and often two to six, carbon atoms or a branched monovalent hydrocarbon moiety of three to twenty, typically three to twelve and often three to six carbon atoms, containing at least one carbon-carbon triple bond. Exemplary alkynyls include, but are not limited to, ethynyl, propynyl, and the like.

“Amine compound” refers to an organic compound comprising a substituent of the formula −NR^{a}R^{b}, where each of R^{a} and R^{b} is independently hydrogen, alkyl, heteroalkyl, haloalkyl, aryl, aralkyl, cycloalkyl, (cycloalkyl)alkyl, heteroaryl, heteroaralkyl, heterocycloalkyl, or (heterocycloalkyl)alkyl. Typically, each of R^{a} and R^{b} is independently hydrogen, alkyl, heteroalkyl, haloalkyl, aryl, aralkyl, cycloalkyl, or (cycloalkyl)alkyl. Often each of each of R^{a} and R^{b} is independently hydrogen, alkyl, heteroalkyl, or haloalkyl. And more often each of R^{a} and R^{b} is independently hydrogen, alkyl, or heteroalkyl. The amine compound can also include heterocyclic amine compounds such as piperazine, imidazole, pyridine, oxazoles, thiazoles, etc. each of which can be optionally substituted. “Monoamine compound” refers to an organic compound having one —NR^{a}R^{b} substituent and “diamine compound” refers to an organic compound having two —NR^{a}R^{b} substituents, where each of R^{a} and R^{b} is independently those defined in this paragraph.

“Alkyl amine compound” refers to a hydrocarbon compound comprising a substituent of the formula —NR^{a}R^{b}, where each of R^{a} and R^{b} is independently hydrogen, alkyl, haloalkyl, aryl, aralkyl, cycloalkyl, or (cycloalkyl)alkyl. Typically, each of R^{a} and R^{b} is independently hydrogen, alkyl, aryl, aralkyl, cycloalkyl, or (cycloalkyl)alkyl. Often each of each of R^{a} and R^{b} is independently hydrogen or alkyl.

“Heteroalkyl amine compound” refers to an amine compound as defined herein in which R^{a} is a heteroalkyl group. In particular, heteroalkyl amine compound refers to an organic compound comprising a substituent of the formula —NR^{a}R^{b}, where R^{a} is heteroalkyl, and R^{b} is hydrogen, alkyl, heteroalkyl, haloalkyl, aryl, aralkyl, cycloalkyl, (cycloalkyl)alkyl, heteroaryl, heteroaralkyl, heterocycloalkyl, or (heterocycloalkyl)alkyl. Typically, R^{a} is heteroalkyl, and R^{b} is hydrogen, alkyl, heteroalkyl, haloalkyl, aryl, aralkyl, cycloalkyl, or (cycloalkyl)alkyl. Often R^{a} is heteroalkyl, and R^{b} is hydrogen, alkyl, heteroalkyl, or haloalkyl. More often R^{a} is heteroalkyl, and R^{b} is hydrogen, alkyl, or heteroalkyl. Still more often R^{a} is heteroalkyl, and R^{b} is hydrogen or alkyl.

“Alkanolamine compound” refers to an amine compound as defined herein in which R^{a} is an alkanol group. In particular, alkanolamine compound refers to an organic compound comprising a substituent of the formula —NR^{a}R^{b}, where R^{a} is alkanol, and R^{b} is hydrogen, alkyl, heteroalkyl, haloalkyl, aryl, aralkyl, cycloalkyl, (cycloalkyl)alkyl, heteroaryl, heteroaralkyl, heterocycloalkyl, or (heterocycloalkyl)alkyl. Typically, R^{a} is alkanol, and R^{b} is hydrogen, alkyl, heteroalkyl, haloalkyl, aryl, aralkyl, cycloalkyl, or (cycloalkyl)alkyl. Often R^{a} is alkanol, and R^{b} is hydrogen, alkyl, heteroalkyl, or haloalkyl. More often R^{a} is alkanol, and R^{b} is hydrogen, alkyl, or heteroalkyl. Still more often R^{a} is alkanol, and R^{b} is hydrogen, alkyl, or alkanol.

“Aryl” refers to a monovalent mono-, bi- or tricyclic aromatic hydrocarbon moiety of 6 to 15 ring atoms which is optionally substituted with one or more, typically one, two, or three substituents within the ring structure. When two or more substituents are present in an aryl group, each substituent is independently selected. Exemplary aryl groups include phenyl and naphthyl. Often an aryl group is an optionally substituted, more often unsubstituted, phenyl group. Exemplary substituents of an aryl group include halide, alkoxy, and alkyl.

“Aralkyl” refers to a moiety of the formula —R^{c}-R^{d} where R^{c} is an alkylene group and R^{d} is an aryl group as defined herein. Exemplary aralkyl groups include, but are not limited to, benzyl, phenylethyl, 3-(3-chlorophenyl)-2-methylpentyl, and the like.

“Cycloalkyl” refers to a non-aromatic, typically saturated, monovalent mono- or bicyclic hydrocarbon moiety of three to ten ring carbons. The cycloalkyl can be optionally substituted with one or more, typically one, two, or three, substituents within the ring structure. When two or more substituents are present in a cycloalkyl group, each substituent is independently selected. Often a cycloalkyl group is a saturated monocyclic hydrocarbon moiety.

“(Cycloalkyl)alkyl” refers to a moiety of the formula —R^{x}-R^{y}, where R^{y} is cycloalkyl, and R^{x} is alkylene or heteroalkylene as defined herein. Typically R^{x} is alkylene.

The terms “halo,” “halogen” and “halide” are used interchangeably herein and refer to fluoro, chloro, bromo, or iodo.

“Haloalkyl” refers to an alkyl group as defined herein in which one or more hydrogen atom is replaced by same or different halo atoms. The term “haloalkyl” also includes perhalogenated alkyl groups in which all alkyl hydrogen atoms are replaced by halogen atoms. Exemplary haloalkyl groups include, but are not limited to, —CH₂Cl, —CF₃, —CH₂CF₃, —CH₂CCl₃, and the like.

“Haloalkylene” refers to a branched or unbranched saturated divalent haloalkyl moiety defined above.

“Heteroalkyl” refers to a branched or unbranched, saturated alkyl moiety containing carbon, hydrogen and one or more heteratoms such as oxygen, nitrogen or sulfur, in place of a carbon atom. Exemplary heteroalkyls include, but are not limited to, 2-methoxyethyl, 2-aminoethyl, 3-hydroxypropyl, 3-thiopropyl, and the like.

“Heteroalkylene” refers to a branched or unbranched saturated divalent heteroalkyl moiety defined above.

The terms “alkanol” and “hydroxyalkyl” are used interchangeably herein and refer to an alkyl group having one or more, typically one, hydroxyl groups (—OH). Exemplary hydroxyalkyls include, but are not limited to, 2-hydroxyethyl, 6-hydroxyhexyl, 3-hydroxyhexyl, and the like.

“Heteroaryl” refers to an aryl group as defined herein in which one or more, typically one or two, and often one, of the ring carbon atom is replaced with a heteroatom selected from O, N, and S.

“Heteroaralkyl” refers to a moiety of the formula —R^{m}-Rⁿ where R^{m} is an alkylene group and Rⁿ is a heteroaryl group as defined herein.

“Hydrocarbon” refers to a linear, branched, cyclic, or aromatic compound having hydrogen and carbon.

“Silyl” and “siloxy” refer to a moiety of the formula —SiR^{e}R^{f}R^{g} and —OSiR^{e}R^{f}R^{g}, respectively, where each R^{e}, R^{f}, and R^{g} is independently hydrogen, alkyl, cycloalkyl, or (cycloalkyl)alkyl or two or more of R^{e}, R^{f}, and R^{g} combine to form a cycloalkyl or (cycloalkyl)alkyl group.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

“Corresponding protecting group” means an appropriate protecting group corresponding to the heteroatom (i.e., N, O, P or S) to which it is attached.

When describing a chemical reaction, the terms “treating”, “contacting” and “reacting” are used interchangeably herein, and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

### Compositions of the Invention

Some aspects of the invention provide compositions comprising an ionic liquid (IL) and an amine compound. Compositions of the invention can also include a solvent. When present, the solvent is typically an organic solvent, water, or a combination thereof. Exemplary organic solvents that can be used with compositions and methods of the invention include, but are not limited to, methanol, ethanol, propanol, glycols, acetonitrile, dimethyl sulfoxide, sulfolane, dimethylformamide, acetone, dichloromethane, chloroform, tetrahydrofuran, ethyl actetate, 2-butanone, toluene, as well as other organic solvents known to one skilled in the art.

Suitable ionic liquids for the compositions of the invention are salts whose melting point is relatively low (e.g., ≦100° C., typically ≦50° C.). The salts that are liquid at room temperature are called room-temperature ionic liquids, or RTILs, which are often used in compositions of the present invention. Typically, any RTIL can be used in compositions of the present invention. Exemplary ionic liquids that are suitable for use in compositions of the present invention include, but are not limited to, imidazolium-based RTILs (see, for example, Anthony et al., Int. J. Environ. Technol. Manage., 2004, 4, 105; Baltus et al., Sep. Sci. Technol., 2005, 40, 525; Zhang et al., AIChE J., 2008, 54, 2717; Finotello et al., J. Phys. Chem. B, 2008, 112, 2335; Kilaru et al., Ind. Eng. Chem. Res., 2008, 47, 910; Kilaru et al., Ind. Eng. Chem. Res., 2008, 47, 900; Anderson et al., Acc. Chem. Res., 2007, 40, 1208; Hou et al., Ind. Eng. Chem. Res., 2007, 46, 8166; Schilderman et al., Fluid Phase Equilibr., 2007, 260, 19; Finotello et al., Ind. Eng. Chem. Res., 2008, 47, 3453; Jacquemin et al., J. Solution Chem., 2007, 36, 967; Shiflett et al., J. Phys. Chem. B, 2007, 111, 2070; Kumelan et al., J. Chem. Thermodyn., 2006, 38, 1396; Camper et al., Ind. Eng. Chem. Res., 2006, 45, 6279; Kumelan et al., J. Chem. Eng. Data, 2006, 51, 1802; Fu et al., J. Chem. Eng. Data, 2006, 51, 371; Shiflett et al., Ind. Eng. Chem. Res., 2005, 44, 4453; Anthony et al., J. Phys. Chem. B, 2005, 109, 6366; Scovazzo et al., Ind. Eng. Chem. Res., 2004, 43, 6855; Cadena et al., J. Am. Chem. Soc., 2004, 126, 5300; Camper et al., Ind. Eng. Chem. Res., 2004, 43, 3049; Baltus et al., J. Phys. Chem. B., 2004, 108, 721; Morgan et al., Ind. Eng. Chem. Res., 2005, 44, 4815; Ferguson et al., Ind. Eng. Chem. Res., 2007, 46, 1369; and Camper et al., Ind. Eng. Chem. Res., 2006, 45, 445), phosphonium-based RTILs (see, for example, Kilaru et al., Ind. Eng. Chem. Res., 2008, 47, 910; Kilaru et al., Ind. Eng. Chem. Res., 2008, 47, 900; and Ferguson et al., Ind. Eng. Chem. Res., 2007, 46, 1369), ammonium-based RTILs (see, for example, Kilaru et al., Ind. Eng. Chem. Res., 2008, 47, 910; Kilaru et al., Ind. Eng. Chem. Res., 2008, 47, 900; and Jacquemin et al., J. Solution Chem., 2007, 36, 967), pyridinium-based RTILs (see, for example, Anderson et al., Acc. Chem. Res., 2007, 40, 1208; and Hou et al., Ind. Eng. Chem. Res., 2007, 46, 8166), sulfonium-based RTILs, oxazolium-based RTILs, thiazolium-based RTILs, triazolium-based RTILs, and tetrazolium-based RTILs. Compositions of the invention can include a single ionic liquid compound or it can be a mixture of two or more different ionic compounds depending on the particular properties desired.

In some embodiments, the ionic liquid is an imidazolium-based IL, typically an imidazolium-based RTIL. Some of the methods for producing imidazolium-based IL are disclosed in a commonly assigned PCT Patent Application entitled “Heteroaryl Salts and Methods for Producing and Using the Same,” which is filed even date herewith. RTILs can be synthesized as custom or “task-specific” compounds with functional groups that enhance physical properties, provide improved interaction with solutes, or are themselves chemically reactive. Multiple points are available for tailoring within the imidazolium-based IL, presenting a seemingly infinite number of opportunities to design ILs matched to individual solutes of interest. Furthermore, many imidazolium-based ILs are miscible with one another or with other solvents; thus, mixtures of ILs serve to multiply the possibilities for creating a desired solvent for any particular application. Separations involving liquids or gases are just one area where the design of selective ILs is of great utility and interest.

In some embodiments, the imidazolium-based IL is of the formula:

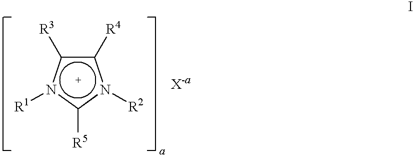

where
a is an oxidation state of X;
X is a counter anion; and
each of R¹ and R² is independently alkyl, heteroalkyl, cycloalkyl, haloalkyl, silyl, siloxyl, aryl, alkenyl, or alkynyl;
each of R³, R⁴, and R⁵ is independently hydrogen, alkyl, cycloalkyl, heteroalkyl, haloalkyl, silyl, siloxyl, aryl, alkenyl, or alkynyl.

Within the imidazolium-based IL of Formula I, in some instances X comprises OTf, BF₄, PF₆, Tf₂N, halide, dicyanamide (dca), or sulfonate. In other instances a is 1. Still in other instances R³, R⁴, and R⁵ are hydrogen. While in other instances at least one of R¹ and R² is alkyl. In other instances at least one of R¹ and R² is heteroalkyl. In some particular embodiments, heteroalkyl is hydroxyalkyl. In some cases, the hydroxyalkyl is C₂₋₆ hydroxyalkyl. In other embodiments, haloalkyl is fluoroalkyl.

Still in other embodiments, each of R¹ and R² is independently alkyl, haloalkyl, or heteroalkyl. Typically each of R¹ and R² is independently alkyl, fluoroalkyl, hydroxyalkyl, or nitrile alkyl (i.e., —R—CN, where R is alkylene). Often each of R¹ and R² is independently alkyl or hydroxyalkyl. More often, one of R¹ and R² is alkyl and the other is hydroxyalkyl.

Yet in other instances the imidazolium-based IL is of the formula:

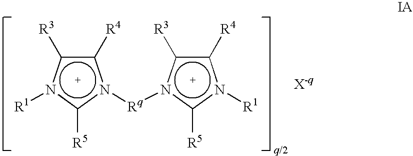

where
q is an oxidation state of X;
each X is independently a counter anion; and
each R¹ is independently alkyl, heteroalkyl, cycloalkyl, haloalkyl, silyl, siloxyl, aryl, alkenyl, or alkynyl;
each of R³, R⁴, and R⁵ is independently hydrogen, alkyl, cycloalkyl, heteroalkyl, haloalkyl, silyl, siloxyl, aryl, alkenyl, or alkynyl; and
R^{q} is alkylene, heteoralkylene, or haloalkylene.

Typically, compounds of Formula IA are RTIL.

Within the imidazolium-based IL of Formula IA, in some instances X comprises OTf, BF₄, PF₆, Tf₂N, halide, or sulfonate. In other instances q is 1. Still in other instances R³, R⁴, and R⁵ are hydrogen. While in other instances at least one of each R¹ is independently alkyl, heteroalkyl or haloalkyl. In other instances at least one of R¹ is heteroalkyl. In some particular embodiments, heteroalkyl is hydroxyalkyl. In some cases, the hydroxyalkyl is C₂₋₆ hydroxyalkyl.

Typically, R^{q} is alkylene.

Still in other embodiments, each R¹ is independently alkyl, fluoroalkyl, hydroxyalkyl, or nitrile alkyl (i.e., —R—CN, where R is alkylene). Often each R¹ is independently alkyl or hydroxyalkyl. More often, one of R¹ is alkyl and the other is hydroxyalkyl.

The compositions of the present invention include an amine compound. In some embodiments, the amine compound is a heteroalkylamine compound. Within these embodiments, in some instances, the amine compound is an alkanolamine compound. Typically, alkanolamine compound comprises a primary amine group. In other instances, the alkanolamine compound comprises a primary hydroxyl group. Typically, the alkanolamine compound comprises C₂-C₁₀ alkyl chain and often C₂-C₆ alkyl chain. However, it should be appreciated the length of the alkyl chain is not limited to these specific ranges and examples given herein. The length of the alkyl chain can vary in order to achieve a particular property desired.

Still in other embodiments, the amine compound is a monoamine compound. In some instances within these embodiments, the monoamine compound is of the formula:

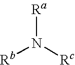

where
each of R^{a} and R^{b} is independently hydrogen, alkyl, aryl, aralkyl, cycloalkyl, (cycloalkyl)alkyl, haloalkyl, heteroalkyl, alkenyl, alkynyl, silyl or siloxyl; and
R^{c} is hydrogen, alkyl, aryl, aralkyl, cycloalkyl, (cycloalkyl)alkyl, haloalkyl, heteroalkyl, alkenyl, alkynyl, silyl, siloxyl, or a nitrogen protecting group.
   Typically, each of R^{a} and R^{b} is independently hydrogen, alkyl, or heteroalkyl; and R^{c} is hydrogen, alkyl, or heteroalkyl. Often the heteroalkyl is hydroxyalkyl. Often the heteroalkyl is hydroxyalkyl. Exemplary hydroxyalkyls include, but are not limited to, 2-hydroxyethyl, 3-hydroxypropyl, 2-hydroxypropyl, 4-hydroxybutyl, 3-hydroxybutyl, 2-hydroxybutyl, and the like. In some particular embodiments, the monoamine compound is selected from the group consisting of mono(hydroxyalkyl)amine, di(hydroxyalkyl)amine, tri(hydroxyalkyl)amine, and a combination thereof. Within these particular embodiments, in some cases the monoamine compound comprises monoethanolamine, diethanolamine, triethanolamine, or a combination thereof. It should be appreciated, however, the compositions of the invention are not limited to these particular monoamine compounds and examples given herein. The scope of the present invention includes other monoamine compound in order to achieve a particular property desired.

Yet in other embodiments, the amine compound is a diamine compound. In some instances within these embodiments, the diamine compound is of the formula:

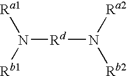

where
each of R^{a1}, R^{a2}, R^{b1}, and R^{b2} is independently hydrogen, alkyl, aryl, aralkyl, cycloalkyl, (cycloalkyl)alkyl, haloalkyl, heteroalkyl, alkenyl, alkynyl, silyl or siloxyl;

R^{c} is hydrogen, alkyl, aryl, aralkyl, cycloalkyl, (cycloalkyl)alkyl, haloalkyl, heteroalkyl, alkenyl, alkynyl, silyl, siloxyl, or a nitrogen protecting group; and

R^{d} is alkylene, arylene, aralkylene, cycloalkylene, haloalkylene, heteroalkylene, alkenylene, alkynylene, silylene or siloxylene.

Typically, each of R^{a1}, R^{a2}, R^{b1}, and R^{b2} is independently hydrogen, alkyl, or heteroalkyl; and R^{c} is hydrogen, alkyl, or heteroalkyl. Often the heteroalkyl is hydroxyalkyl. Exemplary hydroxyalkyls include, but are not limited to, 2-hydroxyethyl, 3-hydroxypropyl, 2-hydroxypropyl, 4-hydroxybutyl, 3-hydroxybutyl, 2-hydroxybutyl, and the like. R^{d} is generally alkylene, typically C₂-C₁₀ alkylene, and often C₂-C₆ alkylene. Exemplary alkylenes include, but are not limited to, ethylene, propylene, butylenes, pentylene, hexylene, 2-methylethylene, 2-methylbutylene, 2-ethylpropylene, and the like. It should be appreciated, however, the compositions of the invention are not limited to these particular diamine compounds and examples given herein. The scope of the present invention includes other diamine compounds in order to achieve a particular property desired.

In other embodiments, the amine compound is an alkyl amine compound including, monoalkyl-, dialkyl-, and trialkylamine compounds. Typically each alkyl group within the alkyl amine compound is independently C₁-C₁₀ alkyl group. Often each alkyl group is independently C₁-C₆ alkyl group, and more often each alkyl group is independently C₁-C₃ alkyl group.

The relative amount of ionic liquid compared to the total amount of ionic liquid and the amine compound can vary widely. It should be appreciated that in general, the impurity or the undesired compound that one wishes to remove from a source forms a complex or an addition product with the amine compound or becomes solubilized in the composition, accordingly the higher amount of the amine compound provides a higher amount of the complex or an addition product formation. Typically when the amine compound is an alkylamine compound, the relative amount of the ionic liquid compound compared to the total amount of the ionic liquid and the amine compound is about 85 vol % or less, often about 60 vol % or less, and more often about 50 vol % or less. Alternatively, the relative amount of the ionic liquid compound compared to the total amount of the ionic liquid and the amine compound is about 85 wt % or less, often about 70 wt % or less, more often about 60 wt % or less, and still more often about 50 wt % or less. It should be appreciated, however, the relative amount of the ionic liquid compared to the total amount of the ionic compound and the amine compound is not limited to these particular ranges and examples given herein. The scope of the present invention includes any relative amount of the ionic liquid compared to the total amount of the ionic compound and the amine compound as long as the composition can be used to remove impurities or undesired material from a source.

When the amine compound is an alkanolamine compound, the relative amount of the ionic liquid compound compared to the total amount of the ionic liquid and the amine compound can be any amount as long as the composition can be used to remove impurities or undesired material from a source. However, as stated herein, when the composition is used to remove or separate one or more impurities and/or undesired materials from a source, the amine compound typically forms a complex or an addition product (“complex product” or “addition product”, respectively) with such impurities and/or undesired materials. Thus, in general the higher amount of the amine compound in the composition provides a higher amount of impurities and/or undesired materials to be removed from the source.

Still further, combinations of various groups described herein form other embodiments. For example, in one particularly embodiment of imidazolium-based IL of Formula I, R¹ is alkyl, a is 1, R² is hydroxyalkyl, and R³, R⁴, and R⁵ are hydrogen. In this manner, a variety of compounds and compositions are embodied within the present invention.

### Utility

Descriptions of well known processing techniques, components, and equipment are omitted so as not to unnecessarily obscure the methods and devices in unnecessary detail. The descriptions of the methods and devices disclosed herein are exemplary and non-limiting. Certain substitutions, modifications, additions and/or rearrangements falling within the scope of the claims, but not explicitly listed in this disclosure, will become apparent to those of ordinary skill in the art based on this disclosure.

The compositions of the invention can be used in a wide variety of application including as catalytic systems in various reactions, extraction media, cleaning composition, as well as other applications for ionic liquids that are known to one skilled in the art. In some embodiments, compositions of the invention are used under pressure. Such increased pressure can increase the rate of complex and/or addition product formation.

When the source is a fluid medium, e.g., a gas or a liquid, compositions of the invention can be used to remove, separate or extract one or more impurities and/or undesired materials from the source. For example, compositions of the invention can be used to remove undesired gas such as CO₂, CO, COS, H₂S, SO₂, NO, N₂O, mercaptans (e.g., alkylmercaptans), H₂O, O₂, H₂, N₂, methane, propane, and other relatively short chain hydrocarbons and/or volatile organic compounds.

Typically, different gases have different solubility depending on the nature of ionic liquids. In some instances, two or more ionic liquids in combination provides higher solubility. Accordingly, the scope of the present invention includes compositions having a mixture of two or more different ionic liquids.

Without being bound by any theory, it is believed that the ionic liquid solubilizes the impurities and the amine compound forms a complex and/or an addition product with the impurities. Accordingly, it is believed that both the ionic liquid and the amine compound are responsible for the effectiveness of removing the impurities. Thus, the selection of the amine compound and the ionic liquid is believed to be important in removing the impurities. Typically, the compositions of the invention are miscible. That is, the amine compound and the ionic liquid do not form a separate layer but form a single miscible layer. In some instances, a solvent can be added to aid in miscibility of the amine compound and the ionic liquid. Typically, the amine compound is also reactive or is capable of relatively readily forming a complex with the impurities. Generally an alkyl amine compound or a heteroalkyl amine compound, in particular an alkanolamine compound, are used in compositions and methods of the invention due to their high reactivity as well as the cost considerations.

In some embodiments, methods for removing the impurities include pressurizing the admixture of compositions of the invention and the source to be purified. It is believed that subjecting the admixture to pressurized conditions (i.e., greater than the standard pressure which is 1 atm) increases the rate of complex and/or addition product formation between the impurities and the amine compound. When pressurizing conditions are used, typically a pressure of greater than 1 atm, more often at least 2 atm, and still more often at least 5 atm is used.

As discussed above, compositions of the invention can be used to remove impurities from a wide variety of sources including, but not limited to, various solids such as semi-conductors and other electronic devices, fluids such as natural gas, waste emission, oil, gases evolved from biological sources, respiratory gases, combustion products, decomposition products, chemical reactions, gases released as a result of depressurization, or any other fluid medium sources in which a removal or separation of undesired gases is desired.

For the sake of clarity and brevity, methods of the invention will now be described with respect to reducing a gas impurity from a fluid medium. However, it should be appreciated that one skilled in the art having read the present disclosure can readily adopt compositions and methods of the invention for removing other impurities from various sources.

Various embodiments of the methods and apparatuses of the invention comprise one or more of the following general components: an impurity removing mixture comprising a composition of the invention, i.e., an ionic liquid and an amine compound. The ionic liquid typically comprises a room temperature ionic liquid. Compositions of the invention can optionally include a solvent, such as water, an organic solvent, or a combination thereof. Exemplary organic solvents that are suitable in methods of the present invention include, but are not limited to, chloroform, dichloromethane, methanol, ethanol, propanol, glycols, acetonitrile, dimethyl sulfoxide, sulfolane, dimethylformamide, acetone, tetrahydrofuran, ethyl acetate, 2-butanone, toluene and other organic solvents known to one skilled in the art.

RTILs have a number of properties that make them useful in gas separations. For example, RTILs are generally non-volatile, largely inflammable, and have good gas (e.g., CO₂) solubility and CO₂/N₂ and CO₂/CH₄ separation selectivity. The dissolution of CO₂ (and other gases) in RTILs (and other solvents) is believed to be a physical phenomenon, with no appreciable chemical reaction occurring unlike with amine solutions that are often used in other methods.

Amine-functionalized RTILs (those containing amine groups chemically tethered to the anion and/or cation) are not feasible for use in a large industrial setting or in smaller-scale CO₂ capture devices, such as those on submarines. The use of these amine-functionalized RTILs as neat (without a co-solvent) solvents for CO₂ capture is an ill-conceived notion. The viscosity of amine-functionalized RTILs used in CO₂ capture is quite high, thereby limiting its implementation in large scale scrubbing applications. Furthermore, amine-functionalized RTILs no longer resemble a liquid upon capture of CO₂, but instead often form an intractable tar.

The present inventors have discovered a cheaper and more attractive method to combine amine compounds and ILs without the use of covalent linkages. Such combination avoids formation of intractable tar which is often the case with an amine tethered RTILs. Inexpensive, commercially used amines, such as monoethanolamine (MEA) or diethanolamine (DEA), can be readily dissolved in ILs. These amine-IL solutions can be used effectively for the capture of various impurities or gases including, but not limited to, CO₂, CO, COS, H₂S, SO₂, NO, N₂O, alkyl mercaptans, H₂O, O₂, H₂, N₂, methane, propane and other relatively short chain hydrocarbons, and volatile organic compounds.

Currently, various aqueous amine solutions are used in various industries to remove CO₂ and/or H₂S. Compositions of the present invention offer significant advantages over their aqueous counterparts, for example, a lower energy usage per volume of CO₂ captured. Furthermore, the ability to tune the IL to enhance the rate of CO₂ uptake and the volume of fluid needed to process the captured CO₂ makes them very attractive as a gas capture media.

The removal of CO₂, H₂S, and other gases from natural gas (e.g., CH₄) and air (including recirculated air) is important to industry, society and the environment. Currently, the separation of CO₂ from other gases is accomplished through its contacting and subsequent reaction with an aqueous amine solution. Typical and widely used water-soluble amine compounds and the pressure of CO₂ where they are effective are shown below:

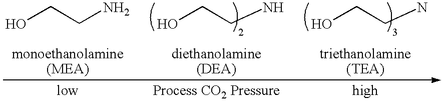

The reaction mechanisms for forming a carbamate salt with MEA is illustrated below:

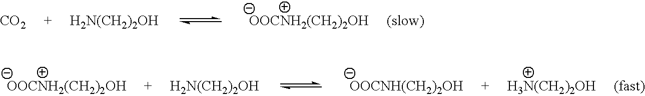

Without being bound by any theory, it is believed that the rate-limiting step of the formation of the zwitterion is maintained by the proton transfer reaction to form a carbamate. The CO₂-adduct remains in aqueous solution and this chemically bound CO₂ remains in the solution unless the solution is heated, the partial pressure is reduced or a combination of both. This process is effective for the separation of CO₂ from other gases on large and small scales.

The present inventors have found that compositions comprising a RTIL and an amine compound (“RTIL-amine solutions”, such as RTIL-MEA) are effective in CO₂ capture in a manner similar to their aqueous counterparts. Such mixtures exhibit rapid and reversible CO₂ uptake, and are capable of capturing 1 mole of CO₂ per 2 moles of dissolved amine.

RTIL-amine solutions offer many advantages over conventional aqueous amine solutions, especially in the energy required to process acid gases (e.g., CO₂). For example, imidazolium-based RTILs have less than one-third the heat capacity of water (e.g., 1.30 vs. 4.18 J g⁻¹ K⁻¹), or less than one-half on a volume basis (e.g., 1.88 vs. 4.18 J cm⁻³ K⁻¹). Decomplexation of CO₂ from aqueous carbamates requires heating the solution to elevated temperatures, where water and some amine need to be condensed or replaced. While alkanolamines have relatively low vapor pressures, it is believed that their volatility is further suppressed due to colligative properties in RTIL solutions, potentially minimizing amine losses. Furthermore, unlike other solvents, both the solubility and selectivity of CO₂ (or any other undesired material) in RTILs can be readily “tuned” by tailoring the structures of the cation and/or anion, or by using one or more additional amine compounds to promote miscibility.

In aqueous solutions, MEA is generally the most commonly used amine compound for low partial pressure acid gas applications. MEA is miscible with both [C₆mim][Tf₂N] and [C₂OHmim][Tf₂N], whose structures are given below, respectively:

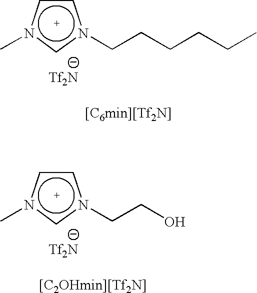

but the corresponding CO₂ adduct, i.e., carbamate shown below:

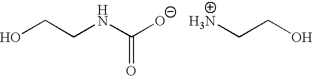

is not soluble in either [C₆mim][Tf₂N] or [C₂OHmim][Tf₂N]. It should also be noted that some amine compounds that are useful for CO₂ capture are not necessarily soluble in every RTIL. For example, DEA was found to be immiscible with RTILs containing solely alkyl substituents (i.e., [C₆mim][Tf₂N]). To expand RTIL-amine solutions to 2° alkanolamines, an RTIL containing a tethered 1° alcohol (e.g., [C₂OHmim][Tf₂N]) was used, which was miscible with MEA and DEA. The ability to tune the solubility and compatibility properties of RTILs is a powerful tool for process optimization, and allows for these solutions to be used for CO₂ capture at a range of pressures. These broad capabilities of RTIL-amine solutions are not easily obtainable with a “task-specific” ionic liquid (TSIL, i.e., an “amine-tethered RTILs”). As the CO₂ adduct carbamate is not soluble in the RTIL solution, the reaction equilibrium is shifted to further favor formation of the carbamate, making it possible to remove even small amounts of CO₂ and H₂S from very dilute gas mixtures. The MEA-based carbamate is not soluble in either [C₆mim][Tf₂N] or [C₂OHmim][Tf₂N], therefore, this reduces the concentration of the carbamate in the solution. By reducing the carbamate concentration in solution the residual CO₂ content in the gas can be brought to very low levels by shifting the proton transfer reaction to the right. The solubility of the carbamate is in sharp contrast to the behavior of these salts in aqueous (or polar organic) solutions. For example, carbamate salts of MEA are highly soluble in water.

As discussed above, the amine compound forms a carbamate with CO₂, as such methods of the invention can also be used in synthesis of carbamates or other addition products between an amine compound and a compound comprising a complementary functional group that is reactive with the amine functional group. Alternatively, by using other functionalized compounds in place of an amine compound, one can achieve synthesis of a wide variety of compounds.

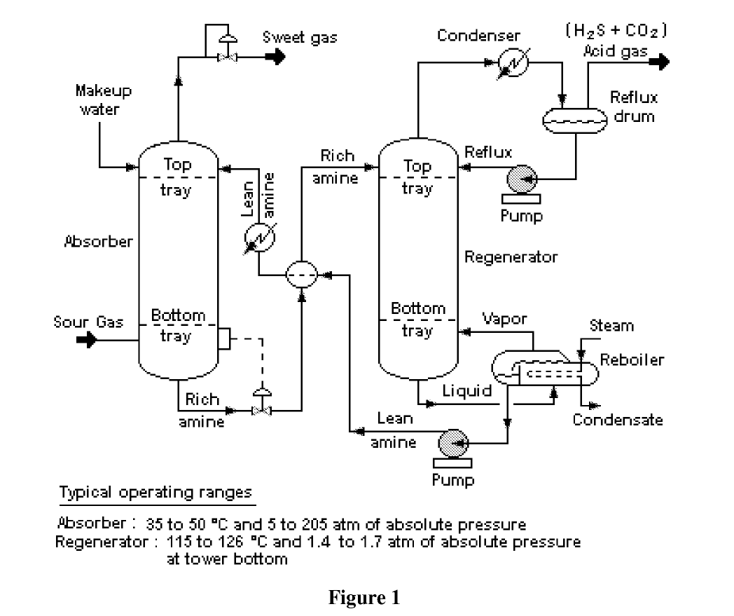

FIG. 1 is a schematic representation of a typical aqueous amine gas treatment unit. RTILs can be utilized in several ways with only minimal modifications to aqueous amine gas treatment unit. One method is to simply replace the solvent (water) with a composition of the present invention. Since many RTILs have approximately half the heat capacity of water on a volume basis, there is an energy savings from the heating and cooling of the solution between the absorber and regenerator. Furthermore, since RTILs have a very low vapor pressure there are no significant losses of the RTIL due to vaporization. Losses of the amine (and a solvent if any is used) are also reduced due to colligative properties where the amine/solvent vapor pressure is reduced due to the low vapor pressure of the RTIL. Another benefit of the low vapor pressure of the RTIL is that if a sweep gas is needed (in typical aqueous amine solutions water vapor is the sweep gas) a more energy efficient method can be implemented. Another way that RTILs can be used to improve energy efficiency is due to the fact that while MEA is soluble in RTILs like [C₆mim][Tf₂N] the corresponding carbamate is not. This allows for the separated carbamate to be regenerated without having the added energy consumption of heating a large volume of solvent to the temperature necessary to regenerate the amine.

It should be appreciated that processes of the invention are not limited to the process shown in FIG. 1. One skilled in the art can readily modify, delete, and/or add various components and/or elements shown in FIG. 1. For example, the process can be a virtually a continuous process or it can be a stepwise process. Furthermore, processes of the invention can also include a pre-mixing step where the amine compound and the ionic liquid is mixed prior to contacting the mixture with the fluid stream. Such a pre-mixing step can be achieved in a separate chamber or the amine compound and the ionic liquid can be injected into the extraction chamber simultaneously through separate inlets (or separately or stepwise through separate inlets or the same inlet) under turbulent conditions, e.g., jet stream, to provide mixing.

The processes of the invention can also include monitoring the extraction (e.g., removal of impurity). For example, one can monitor the amount of the amine compound present in the mixture and provide addition of additional amount of the amine compound as needed. Such processes can be automated using a system comprising a central processing unit (e.g. a computer or other similar devices). Monitoring the amine compound in the mixture can be achieved by any of the analytical processes known to one skilled in the art. For example, one can sample the mixture at a pre-determined intervals or randomly and analyze the mixture for the presence of the amine compound. Alternatively, the amine compound can be monitored continuously, for example, by providing a sampling window within the extraction vessel that allows monitoring of the amount of the amine compound by a suitable analytical technique such as, but not limited to, infrared analysis, UV/Vis analysis, nuclear magnetic resonance (NMR), etc. In this manner, a relatively constant or steady state level of the amine compound can be maintained within the extraction vessel.

Methods of the invention are suitable for removing various impurities (e.g., gases such as acid gases) from any fluid medium including, but not limited to, gaseous emission streams that comprise an acid gas or undesired gas, gases from natural sources as well as industrial emissions, and oil. Exemplary industries that produce a significant amount of acid gas that can be removed by methods of the invention include, but are not limited to, the energy industry (such as oil refineries, the coal industry, and power plants), cement plants, and the auto, airline, mining, food, lumber, paper, and manufacturing industries.

Some of the natural sources of CO₂ include the byproduct of metabolism, combustion or decay of an organism. In these instances, such sources can produce CO₂ with a carbon isotope make-up different from that of manufactured CO₂. For example, CO₂ from a natural source (e.g., wellhead, combustion of a fossil fuel, respiration of a plant or animal, or decay of garbage, etc.) would have a carbon isotope ratio that was relatively higher in ¹⁴C and/or ¹³C versus ¹²C. Such sources provide addition products from the CO₂ (e.g., carbamate) that are enriched in ¹⁴C and/or ¹³C relative to ¹²C. Compounds that are enriched in ¹⁴C and/or ¹³C are useful products in a variety of applications including, but not limited to, (i) general research uses that track carbon in vivo; (ii) diagnostic and research imaging technologies that could identify the new compound from in vivo background, such as MRI (e.g., in vivo tumor detection). Accordingly, the present invention also provides methods for using a natural CO₂ source and products (e.g., carbamate) created using such natural CO₂ sources that have enriched ¹⁴C and/or ¹³C isotopes.

Additional objects, advantages, and novel features of this invention will become apparent to those skilled in the art upon examination of the following examples thereof, which are not intended to be limiting.

### EXAMPLES

### Materials and General Procedures

All syntheses and manipulations were performed in air. All chemicals were purchased from Sigma-Aldrich (Milwaukee, Wis.), with the exception of lithium bis(trifluoromethane)sulfonamide (LiTf₂N), which was obtained from 3M (St. Paul, Minn.). All chemicals were obtained in the highest purity grade possible from these suppliers, and were used as received. All gases including CO₂ were of at least 99.99% purity and purchased from Air Gas (Radnor, Pa.).

### Instrumentation

¹H NMR data were obtained using a Varian INOVA 400 Spectrometer (400 MHz). Water content (ppm) in [C₆mim][Tf₂N] and [C₂OHmim][Tf₂N] was determined using a Mettler Toledo DL32 Karl Fischer coulometer. A Thermolyne MaxiMix Plus vibrating mixer was used for homogenizing RTIL-amine solutions. The stainless steel cell used in CO₂ uptake experiments was custom fabricated. Pressure sensors (PX303) were purchased from Omega. Automated data acquisition was performed using LabView (National Instruments) interfaced through a custom system.

### Synthesis of 1-hexyl-3-methylimidazolium bis(trifluoromethane)sulfonamide (2a)

1-Methylimidazole (103.50 g, 1.2605 mol) was dissolved in CH₃CN (500 mL) in a 1-L round-bottom flask. 1-Bromohexane (228.98 g, 1.3872 mol) was then added, and the reaction mixture heated at reflux for 16 h. The reaction was then stopped, the solvent removed by rotary evaporation, and Et₂O (300 mL) added, resulting in the formation of two phases. The denser, oily phase was stirred in Et₂O for several hours at ambient temperature. Both phases were then poured into deionized H₂O (1 L), and the aqueous phase was then separated from the Et₂O phase. The aqueous phase was washed with EtOAc (3×500 mL) and then collected in a 2-L round-bottom flask. LiTf₂N (398.21 g, 1.3871 mol) was added to the aqueous phase, and an oily phase immediately separated. The mixture was subsequently vigorously stirred for 24 h to ensure thorough mixing in this large vessel. After this time, the oily phase was extracted into CH₂Cl₂ (750 mL) and washed with deionized H₂O (4×500 mL). The fifth aqueous washing was exposed to AgNO₃, to confirm that residual bromide anion was no longer present via the lack of AgBr precipitate formation. The organic phase was then dried over anhydrous MgSO₄, treated with activated carbon, and filtered through a plug of basic Al₂O₃. The solvent was then removed by rotary evaporation, and the final product was dried while stirring at 65° C. under dynamic vacuum (<1 torr) for 16 h. The product 2a was obtained as a clear pale yellow oil. Yield: 464.05 g (82%). The water content in the product was found to be 217 ppm by Karl-Fischer titration.

### Synthesis of 1-(2-hydroxyethyl)-3-methylimidazolium bis(trifluoromethane)sulfonimide (2b)

1-Methylimidazole (77.63 g, 0.9454 mol) was dissolved in CH₃CN (200 mL) in a 1-L round-bottom flask. 2-Chloroethanol (114.12 g, 1.4174 mol) was then added, and the reaction stirred at reflux for 72 h. After this time, the reaction was stopped, and the solvent removed via rotary evaporation. Et₂O (500 mL) was then added, resulting in the formation of two phases. The mixture was then placed in a freezer at −10° C. Upon cooling for several hours, colorless crystals formed. These crystals were then collected, washed with Et₂O (1 L), and dried at ambient temperature under dynamic vacuum (<1 torr) overnight, yielding 124.35 g (81%) of 1-(2-hydroxyethyl)-3-methylimidazolium chloride. The 1-(2-hydroxyethyl)-3-methylimidazolium chloride (50.00 g, 0.3110 mol) was then dissolved in deionized H₂O (300 mL), to which LiTf₂N (89.28 g, 0.3110 mol) was added to immediately form a separated oily phase. The reaction was then stirred overnight at ambient temperature, after which the oily phase was extracted with EtOAc (500 mL) and washed with deionized H₂O (4×250 mL). The absence of chloride anion was confirmed through addition of AgNO₃ to the fourth aqueous washing, without any AgCl precipitate formation. The organic phase was then dried over anhydrous MgSO₄, treated with activated carbon, and filtered through a plug of basic Al₂O₃. The solvent was removed via rotary evaporation, and the product dried under dynamic vacuum (<1 torr) while stirring at 65° C. overnight to produce 2b as a clear, colorless oil. Yield: 60.58 g (48%). The water content in the product was found to be 225 ppm by Karl-Fischer titration.

### General Procedure for the Formulation of RTIL-Amine Solutions

Solutions of RTILs with amines (50:50 (mol:mol)) were prepared for comparison with amine-functionalized TSILs, which contain one 1° amine group per ion pair. RTIL 2a (10.00 g, 22.35 mmol) was mixed with MEA (1.365 g, 22.35 mmol) in a 20-mL glass vial. The vial was sealed and the liquids were held on a vibrating mixer, typically for <10 s, until a homogeneous solution was achieved. This procedure was repeated for 2b-MEA and 2b-DEA.

### Preparing RTIL-Amine Mixtures With >50 mol % Amine Content

RTILs 2a and 2b were miscible with MEA in all proportions. Solutions containing >50 mol % MEA content were prepared in the same manner as those with 50 mol % content, as outlined above. No phase separation was observed at for any mixture with >50 mol % of MEA. Similarly, 2b was miscible with DEA, and solutions of 2b-DEA with >50 mol % DEA were also prepared. MEA is typically dissolved in water at 30 wt % (˜5 mol/L) in industrial processes.

### CO₂ Capture

CO₂ uptake experiments in RTIL-amine solutions were performed using a dual-volume, dual-transducer apparatus. Briefly, an aliquot of RTIL-amine solution of known mass and volume was sealed in a stainless steel cell of known volume. The cell was heated to 40° C. and purged under dynamic vacuum (<10 torr) for a short time to remove any residual air from the system. CO₂ was then introduced at ˜1 atm. As the CO₂ reacted with the amines, the pressure in the cell was observed to decay and was recorded electronically as a function of time. The difference between the initial and final CO₂ pressures was converted into moles of CO₂ reacted with amine using the ideal gas equation:

$${n}_{{CO}_{2}} = \frac{Δ PV}{RT}$$

Complexation and decomplexation of CO₂ from amines were performed at 40° C. and 100° C.
CO₂ Capture and Release With Equimolar 2a-MEA Solutions

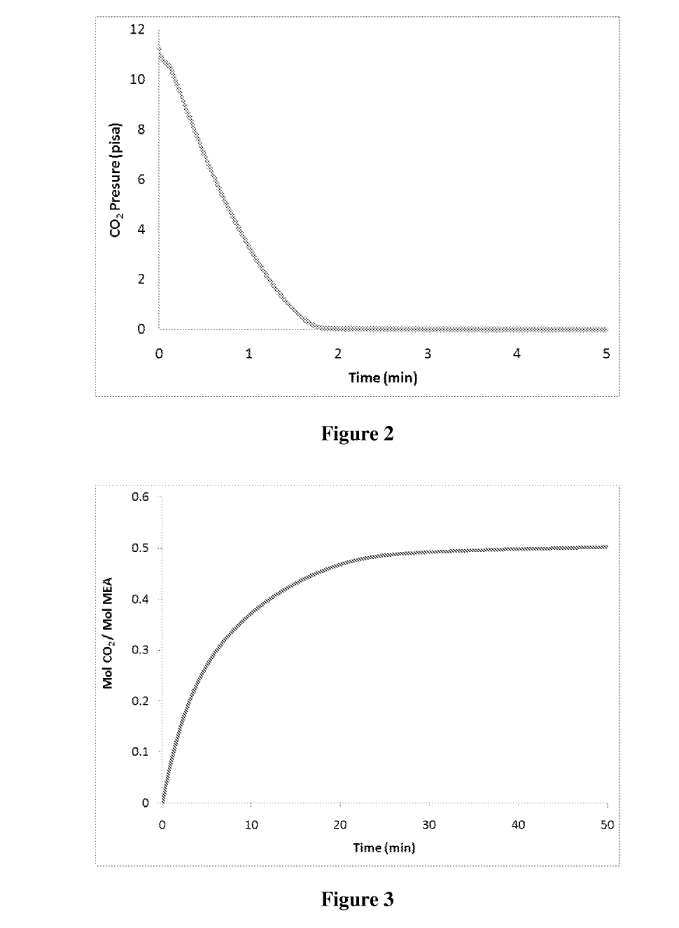

FIG. 2 is an example of the pressure decay of CO₂ in an equimolar solution of 2a-MEA. FIG. 2 shows that the CO₂ concentration in the gas feed was rapidly reduced and effectively brought to zero using an equimolar 2a-MEA solution. These solutions can be rapidly stirred to increase the reaction rate. The final pressure of CO₂ in FIG. 2 is 0±0.015 psia, where 0.015 psia is the accuracy limit of the pressure sensor used. The reaction of CO₂ was favored by MEA-carbamate precipitating from the RTIL solutions.

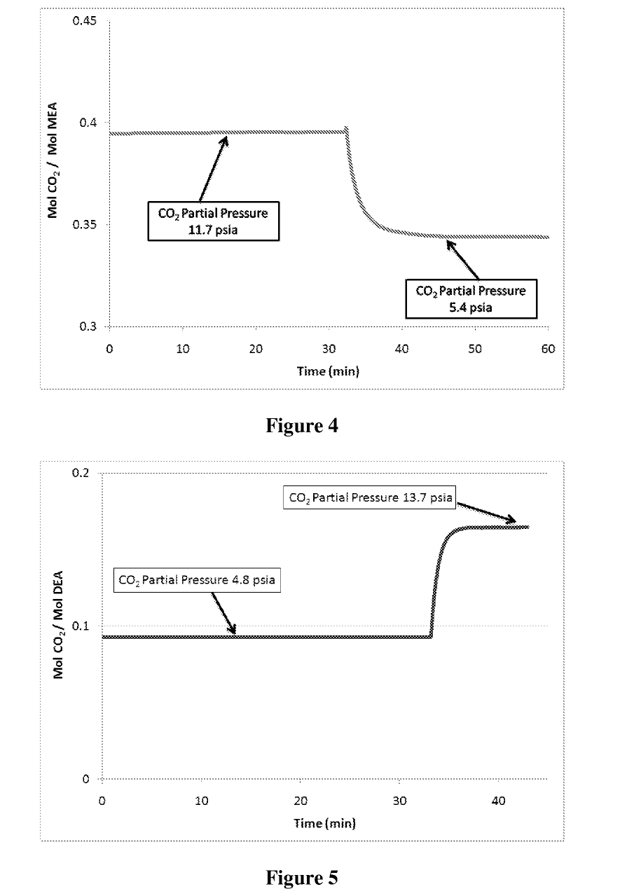

FIG. 3 shows the rate of conversion of CO₂ to MEA-carbamate salt of this system. CO₂ was decomplexed from MEA-carbamate by increasing the temperature to from 40° C. to 100° C. and reducing the pressure from 605 torr (11.7 psia) to 279 torr (5.4 psia), which favors the release of CO₂ and reformation of neutral MEA. FIG. 4 shows the rate of CO₂ release from MEA-carbamate in 2a. Upon reducing the system pressure, to remove some CO₂ from the cell volume, the ratio of CO₂ to amines was reduced from 0.395 to 0.350 within 2 minutes. The initial value of 0.395 is less than 0.500 that was achieved from complete capture at 40° C. This is a consequence of heating from 40° C. to 100° C., as some CO₂ had already been released.

CO₂ Capture and Release With Equimolar 2b-DEA Solutions

CO₂ reacts with DEA in 2b with CO₂ at low pressure to loading levels similar to what can be achieved in aqueous solutions. However, it is believed that DEA-carbamate is a weaker CO₂-adduct than MEA-carbamate, thus the moles of CO₂ captured by DEA are less than 1:2 at the equilibrium pressure of 30.4 torr (0.588 psia). An equilibrium pressure of ˜155 torr (3 psia) was required to achieve a 1:2 ratio of CO₂:DEA.

An added benefit of the 2b-DEA solutions is that increasing the partial pressure of CO₂, even at elevated temperatures, resulted in increased uptake of CO₂ by equimolar 2b-DEA solutions. See FIG. 5. The molar ratio of CO₂ to DEA increased from 0.093 to 0.165 with increasing CO₂ partial pressure from 248 torr (4.8 psia) to 708 torr (13.7 psia) at 100° C. Although aqueous amine solutions are near their boiling points at this temperature, RTILs are effectively non-volatile at 100° C.

### Solubility of Various Gases in IL

1-Ethyl-3-methylimidazolium tetrafluoroborate ([C₂mim][BF₄]) and 1-ethyl-3-methylimidazolium bis-(trifluoromethanesulfonyl)imide ([C₂mim][Tf₂N]) were synthesized according to the procedures described herein. Physical constants of the RTILs (pure and mixtures) are shown in Table 1. The densities of [C₂mim][BF₄] and [C₂mim][Tf₂N] were measured. The average densities of the RTIL mixtures were measured. These RTILs were readily miscible in each other when mixed, and represent a range of molar volumes.

TABLE 1
Physical Properties of Room-Temperature
Ionic Liquids Used in This Study

| | mol. weight | density | molar volume |
|---|---|---|---|
| ionic liquid | (g/mol) | (g/cm³) | (cm³/mol) |
| [C₂mim][Tf₂N] | 391 | 1.50 | 261 |
| 25 mol % [C₂mim][BF₄] | 343 | 1.52 | 226 |
| 50 mol % [C₂mim][BF₄] | 295 | 1.48 | 199 |
| 75 mol % [C₂mim][BF₄] | 246 | 1.42 | 174 |
| 90 mol % [C₂mim][BF₄] | 217 | 1.35 | 161 |
| 95 mol % [C₂mim][BF₄] | 208 | 1.30 | 159 |
| [C₂mim][BF₄] | 198 | 1.28 | 155 |

Additionally, experimental observations and RST have shown that all gases of interest have higher solubility in [C₂mim][Tf₂N] and lower solubility in [C₂mim][BF₄]. However, the solubility selectivity for CO₂ with respect to N₂ and CH₄ is higher in [C₂mim][BF₄] than in [C₂mim][Tf₂N]. These experiments will examine how the combination of the two RTILs properties affect gas solubility behaviors and how to extend regular solution theory (RST) to describe these behaviors in RTIL mixtures.

To determine if the gas-liquid system equilibrium had been reached, the pressure in the cell volume was plotted as a function of time (one measurement per min). After 30 min of constant pressure readings, it was assumed that equilibrium had been reached. All trials displayed similar pressure change behaviors. For each trial, the Henry's constant (“H_{c}”) was determined from the ideal gas law using the difference between Pₜ₌₀ and P_{equil} at each temperature.

Table 2 shows the experimental Henry's constants for each gas/RTIL mixture combination. The Henry's constant for CO₂ and CH₄ increased with increasing [C₂mim][BF₄] content. The Henry's constant for N₂ increased with increasing [C₂mim][BF₄] content, except in pure [C₂mim][BF₄], where the Henry's constant decreased.

TABLE 2
Gas Solubility Trends in RTIL Mixtures

| ionic liquid | CO₂/H_{c} (atm) | N₂/H_{c} (atm) | CH₄/H_{c} (atm) |
|---|---|---|---|
| [C₂mim][Tf₂N] | 50 ± 1 | 1200 ± 60 | 560 ± 10 |
| 25 mol % [C₂mim][BF₄] | 58 ± 3 | 1700 ± 60 | 740 ± 10 |
| 50 mol % [C₂mim][BF₄] | 65 ± 1 | 2400 ± 100 | 980 ± 20 |
| 75 mol % [C₂mim][BF₄] | 85 ± 5 | 4000 ± 600 | 1600 ± 20 |
| 90 mol % [C₂mim][BF₄] | 91 ± 1 | 4500 ± 350 | 1800 ± 60 |
| 95 mol % [C₂mim][BF₄] | 94 ± 1 | 5000 ± 300 | 1900 ± 20 |
| [C₂mim][BF₄] | 100 ± 2 | 3800 ± 100 | 2000 ± 200 |

RST dictates that for low pressure systems, where Henry's law is applicable, gas solubilities (Henry's constant, H₁) can be described by solubility parameters using eq 1 for both the solute and the pure solvent (1=RTIL, 2=gas) and where a and b are empirically determined constants (depending on gas being used and temperature).

ln [H_{2,1}]=a+b(δ₁−δ₂)² (1)

The solubility parameter (δ₁) for pure imidazolium-based RTILs can be estimated using the Kapustinskii equation for lattice energy density and the definition of a solubility parameter. This substitution results in a solubility parameter that is a function of pure RTIL molar volume (eq 2).

δ₁ ∝ [1/(V₁^{4/3})]^{1/2} (2)

Specifically for mixtures, RST states that a volume fraction averaged solubility parameter (δ₁), and related volume fraction averaged molar volume (V₁) for the solvent be used in theoretical calculations (eqs 3 and 4), where is φᵢ the volume fraction and Vᵢ of each pure solvent.

$$\begin{matrix}{\overline{δ}}_{1} = {\sum }_{i} {ϕ}_{i} {δ}_{i} & ( 3 ) \\ {\overline{V}}_{1} = {\sum }_{i} {ϕ}_{i} {V}_{i} & ( 4 )\end{matrix}$$

By combining eqs 1 and 2, the RST model results in eqs 5 and 6, where α and β or β* are experimentally determined constants that are dependent on the temperature and gas being tested.

$$\begin{matrix}ln ( {H}_{2 , 1} ) = α + {β ( {δ}_{1} )}^{2} & ( 5 ) \\ ln ( {H}_{2 , 1} ) = α + ( \frac{{β}^{*}}{{Vi}^{4 / 3}} ) & ( 6 )\end{matrix}$$

It has shown that lower molar volumes tend to have higher ideal solubility selectivities for CO₂. However, in general the theory is less accurate in the low molar volume range.

To determine if the mixtures can be described by RST, a plot was made of the Henry's constant versus the volume fraction average molar volume of the RTIL mixtures, as dictated by RST (eqs 3 and 4). However, use of the volume fraction average mixture molar volume did not result in a quality linear fit for the RST model, which indicated that RST was not a perfect model. Without being bound by any theory, it is believed that this was due to the physical volume change that resulted from mixing the two RTILs. The measured mixture molar volume was not the same as the volume fraction average mixture molar volume (2-6% difference between the measured and calculated values). The difference in the mixture molar volumes indicated that RST was not a robust model; however, using the measured mixture molar volume (empirical data) and the RST equations allowed for the investigation of gas solubility trends in RTILs. Therefore, the average measured mixture molar volume was used in the following plots because it allowed for a more accurate description of the experimentally observed behaviors while using the RST model. For the case of an unknown mixture molar volume, however, it would still be possible to use the volume fraction average mixture molar volume from the known pure component molar volume to get an initial estimate for the gas solubility behavior being investigated. While RST is not exact, it can be used to obtain initial predictions for gas solubilities in new RTILs.

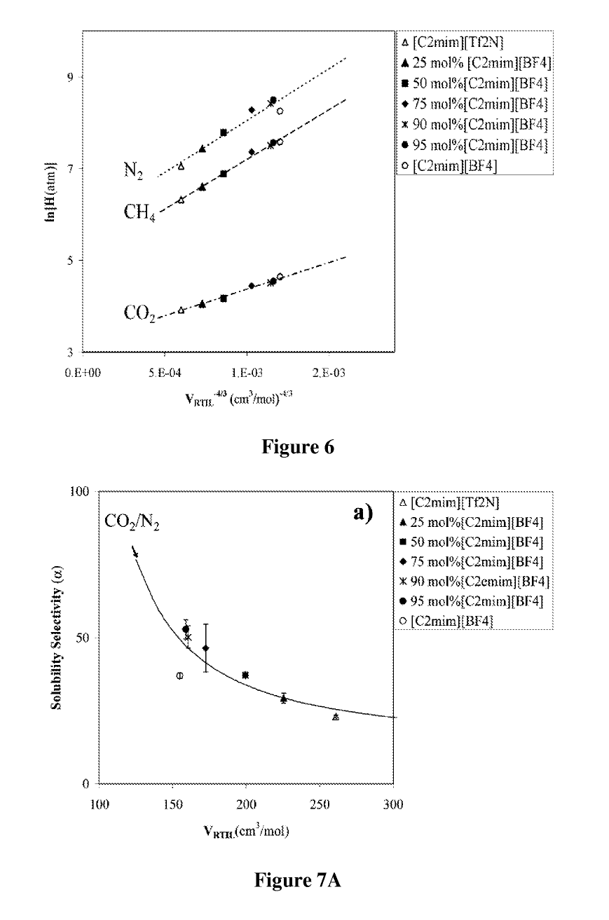

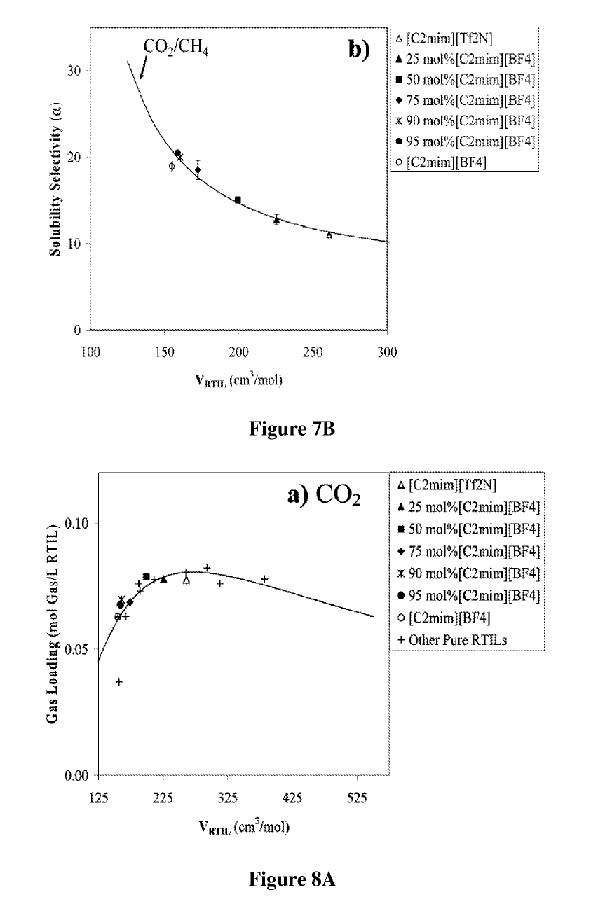

FIG. 6 shows a linear trend for the natural log of the Henry's constants for each gas with respect to average measured mixture molar volume at 40° C. All data shown, including mixtures and pure components, were within the 95% confidence intervals (not shown) of the theoretical line. RST was thus valid for the gas/RTIL mixtures combinations that were investigated. Since RST was valid for these systems, it was expected that lower mixture molar volumes would result in the higher solubility selectivity as shown in FIG. 7. As can be seen, he mixture solubility selectivity agreed with the theoretical line, indicating that RST can be used to describe the behavior of RTIL mixtures using measured molar volumes. All data shown were within the 95% confidence intervals (not shown) of the model. The pure [C₂mim][BF₄] solubility selectivity for both CO₂/N₂ and CO₂/CH₄ did not as closely agree (as compared with the other mixtures and [C₂mim][Tf₂N]) with the theoretical prediction, whereas the 90 and 95 mol % [C₂mim][BF₄] mixtures, at the lower molar volume range of this study, possessed the higher solubility selectivity closer to the RST prediction. Addition of a small amount of [C₂mim][Tf₂N] to [C₂mim][BF₄] resulted in an improved solubility selectivity behavior closer to the theoretical prediction.

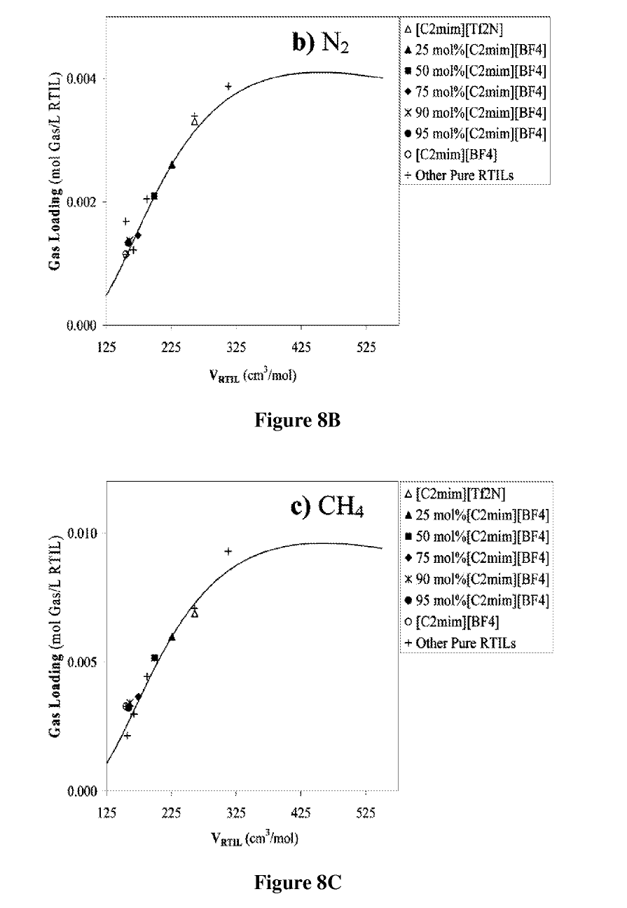

For each gas, the gas loading at 1 atm, or mole fraction of gas dissolved in the RTIL that is in equilibrium with vapor phase, was also examined. FIGS. 8A-C show the results for each gas. These plots used the theoretical parameters to show that the pure component theory could be extended to describe the mixture data. The pure component data for CO₂ includes the following RTILs: 1-butyl-3-methylimidazolium hexafluorophosphate ([C₄mim][PF₆]), 1-butyl-3-methylimidazolium tetrafluoroborate ([C₄mim][BF₄]), 1-butyl-3-methylimidazolium bis[(trifluoromethyl)sulfonyl]imide ([C₄mim][Tf₂N]), 1,3-dimethylimidazolium methylsulfate ([C₁mim][MeSO₄]), 1-hexyl-3-methylimidazolium bis[(trifluoromethyl)sulfonyl]amide ([C₆mim][Tf₂N]), 1-ethyl-3-methylimidazolium trifluoromethanesulfonate ([C₂mim][CF₃SO₃]), 1-ethyl-3-methylimidazolium dicyanamide ([C₂mim][dca]), 1-decyl-3-methylimidazolium trifluoromethanesulfonate ([C₁₀mim][Tf₂N]), [C₂mim][BF₄], and [C₂mim][Tf₂N]. The pure component data for N₂ and CH₄ included the following RTILs: 1,3-dimethylimidazolium methylsulfate ([C₁mim][MeSO₄]), 1-hexyl-3-methylimidazolium bis[(trifluoromethyl)sulfonyl]amide ([C₆mim][Tf₂N]), 1-ethyl-3-methylimidazolium trifluoromethanesulfonate ([C₂mim][CF₃SO₃]), 1-ethyl-3-methylimidazolium dicyanamide ([C₂mim][dca]), [C₂mim][BF₄], and [C₂mim][Tf₂N]. A summary of the pure component data is shown in Table 3.

TABLE 3
Gas Loading at 1 atm and 40° C. for Various Pure RTILs.

| | | gas loading at 1 atm | | |
|---|---|---|---|---|
| | molar | (mol gas/RTIL) | | |
| ionic liquid | volume (cm³/mol) | CO₂ | N₂ | CH₄ |
| [C₁mim][MeSO₄] | 157 | 0.037 | 1.1E−03 | 2.1E−03 |
| [C₂mim][dca] | 167 | 0.063 | 1.2E−03 | 3.0E−03 |
| [C₂mim][CF₃SO₃] | 188 | 0.076 | 2.1E−03 | 4.43-03 |
| [C₄mim][BF₄] | 189 | 0.073 | | |
| [C₄mim][PF₆] | 211 | 0.078 | | |
| [C₄mim][Tf₂N] | 293 | 0.082 | | |
| [C₆mim][Tf₂N] | 313 | 0.076 | 3.9E−03 | 9.3E−03 |
| [C₁₀mim][Tf₂N] | 382 | 0.078 | | |

All mixture data points agreed well (within the 95% confidence intervals) with the theoretical predictions for pure RTILs, and each gas exhibited a maximum gas loading at different molar volumes.

The experimental results indicated that the behavior of gases in RTIL mixtures at constant temperature and low pressure obey RST. Solubility selectivity for CO₂ with N₂ and CH₄ was higher in the 90 and 95 mol % mixtures of [C₂mim][BF₄] in [C₂mim][Tf₂N] than in both pure components or the other mixtures. These two mixtures represent the RTIL mixtures with the smaller molar volumes in this study, and the solubility selectivity was higher than in pure [C₂mim][BF₄], which has an even lower molar volume. These data showed that RST can be used in RTIL mixtures using the average measured molar volume of the mixture. The results showed that RTIL mixtures can be used to enhance CO₂ solubility selectivity due to the control over RTIL molar volume. CO₂ was more soluble gas compared to N₂ or CH₄ in RTIL mixtures tested. Each gas exhibited a maximum gas loading at 1 atm at a different molar volume.

### Mixture of Amine Compounds

Mixtures of different ionic liquids (ILs) and different amines can be varied to tailor performance to different pressures and gas compositions. By using a combination of different ILs the solubility and solubility selectivity of gases can be adjusted (as shown above). This property of ILs can then be applied to adjust reaction rates and reduce other undesirable gas solubility (e.g., hydrocarbon solubility for natural gas sweetening or oxygen solubility for flue gas) for IL/amine applications. A combination of different amines (e.g., MEA and MDEA) in IL/amine applications can be use to adjust the point of carbamate precipitation or prevent carbamate precipitation depending on the ratio. This has many advantages which include control of viscosity, reaction rate, amine acid gas loading, heat of reaction, and corrosion.

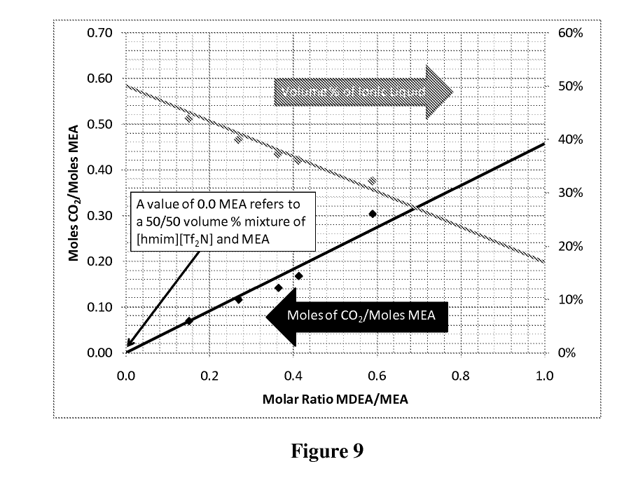

FIG. 9 shows an example of using more than one amine in an IL/amine solution. An initial solution of 50 volume % MEA and 50% volume % [C₆mim][Tf₂N] was made. When the solution was exposed to CO₂ there was immediate carbamate precipitation. Methyldiethanolamine was then added to the solution to act as a proton acceptor, which increased the carbamate solubility forming a homogenous solution. The solution was once again exposed to CO₂ and carbamate precipitation occurred at an elevated amine acid gas loading. Additional MDEA was added and then the process was repeated. The results are shown in FIG. 9 where the black line shows the point of precipitation and the grey line shows the volume percent of IL in the solution. By controlling the point of precipitation, reaction rate can be controlled independently of acid gas loading and acid gas pressure equilibrium.

In addition to imidazolium-based ILs, amines are also miscible in pyridinium-based ILs and phosphonium-based ILs.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for reducing the amount of an impurity gas from a fluid stream, said method comprising contacting the fluid stream with an impurity removing mixture comprising:
an ionic liquid; and
an amine compound,
under conditions sufficient to reduce the amount of impurity gas from the fluid source.

2. The method of claim 1, wherein said method forms an addition product or a complex between the amine compound and the impurity gas.

3. The method of claim 2, wherein the complex or the addition product between the amine compound and the impurity gas forms a precipitate from the impurity removing mixture.

4. The method of claim 1, wherein the impurity removing mixture further comprises a solvent.

5. The method of claim 1, wherein the amine compound comprises a monoamine compound of the formula: 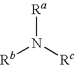
or a diamine compound of the formula: 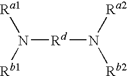
wherein
each of R^{a}, R^{a1}, R^{a2}, R^{b}, R^{b1}, and R^{b2} is independently hydrogen, alkyl, aryl, aralkyl, cycloalkyl, haloalkyl, heteroalkyl, alkenyl, alkynyl, silyl or siloxyl;
R^{c} is hydrogen, alkyl, aryl, aralkyl, cycloalkyl, haloalkyl, heteroalkyl, alkenyl, alkynyl, silyl, siloxyl, or a nitrogen protecting group; and
R^{d} is alkylene, aryl, aralkyl, cycloalkyl, haloalkyl, heteroalkyl, alkenyl, alkynyl, silyl or siloxyl.

6. The method of claim 5, wherein the monoamine compound is selected from the group consisting of mono(hydroxyalkyl)amine, di(hydroxyalkyl)amine, tri(hydroxyalkyl)amine, and a combination thereof.

7. The method of claim 6, wherein the monoamine compound comprises monoethanolamine, diglycolamine, diethanolamine, diisopropylamine, triethanolamine, methyldiethanolamine or a combination thereof.

8. The method of claim 1, wherein the IL is a room temperature ionic liquid (RTIL).

9. The method of claim 8, wherein the RTIL is an imidazolium-based RTIL.

10. The method of claim 1, wherein the impurity gas comprises CO₂, CO, COS, H₂S, SO₂, NO, N₂O, alkyl mercaptans, H₂O, O₂, H₂, N₂, C₁-C₈ chain hydrocarbons, other volatile organic compounds, or a combination thereof.

11. A composition comprising an ionic liquid (IL) and a heteroalkylamine compound.

12. The composition of claim 11, wherein said ionic liquid is a room temperature ionic liquid (RTIL).

13. The composition of claim 11, wherein said ionic liquid comprises an imidazole core structure moiety.

14. The composition of claim 11, wherein said heteroalkylamine compound is an alkanolamine compound.

15. A composition comprising an ionic liquid and an amine compound, wherein the relative volume % of said ionic liquid compared to the total volume of said ionic liquid and said amine compound is about 60 vol % or less.

16. The composition of claim 15, wherein the relative volume % of said ionic liquid is about 50 vol % or less.

17. The composition of claim 15, wherein said amine compound is a heteroalkylamine compound.

18. The composition of claim 17, wherein said heteroalkylamine compound is an alkanolamine compound.

19. A method for removing an impurity from a fluid medium to produce a purified fluid stream, said method comprising contacting the fluid medium with an impurity removing mixture, wherein the impurity removing mixture comprises:
an ionic liquid; and
an amine compound,
under conditions sufficient to remove the impurity from the fluid medium to produce a purified fluid stream.

20. The method of claim 19, wherein the impurity removing mixture further comprises a solvent.

21. The method of claim 19, wherein the ionic liquid is a room temperature ionic liquid (RTIL).

22. The method of claim 19, wherein the impurity is selected from the group consisting of CO₂, CO, COS, H₂S, SO₂, NO, N₂O, H₂O, O₂, H₂, N₂, a volatile organic compound, and a combination thereof.

23. The method of claim 22, wherein the volatile organic compound comprises an organothiol compounds, hydrocarbon, or a mixture thereof.

24. The method of claim 22, wherein the impurity comprises CO₂, SO₂, H₂S, or a combination thereof.

25. The method of claim 19, wherein the amine compound comprises a heteroalkylamine compound.

26. The method of claim 25, wherein the heteroalkylamine compound comprises alkanolamine compound.

27. The method of claim 19, wherein the relative volume % of the ionic liquid relative to the total volume of ionic liquid and the amine compound is about 60 vol % or less.

28. The method of claim 19, wherein said step of contacting the fluid medium with the impurity removing mixture is conducted under pressure.

29. The method of claim 19, wherein the fluid medium comprises a hydrocarbon source.

30. The method of claim 29, wherein the hydrocarbon source comprises natural gas, oil, or a combination thereof.

31. The method of claim 19, wherein said step of contacting the fluid medium with the impurity removing mixture produces an addition product between the impurity and the amine compound.

32. The method of claim 19, wherein said step of contacting the fluid medium with the impurity removing mixture produces a complex between the impurity and the amine compound.

33. The method of claim 19, wherein said step of contacting the fluid medium with the impurity removing mixture solubilizes the impurity in the impurity removing mixture.

34. A method for removing an impurity from a solid substrate surface to produce a clean solid substrate surface, said method comprising:
contacting the solid substrate surface with an impurity removing mixture, wherein the impurity removing mixture comprises:
an ionic liquid; and
an amine compound,
under conditions sufficient to remove the impurity from the solid substrate surface to produce a clean solid substrate surface.

35. The method of claim 34, wherein the solid substrate comprises a semi-conductor.
